(19) United States
(12) Patent Application Publication
(10) Pub. No.: US 2021/0046276 A1
(43) Pub. Date: Feb. 18, 2021

(54) MOOD AND MIND BALANCING AUDIO SYSTEMS AND METHODS

(71) Applicant: Gregory J. Papania, Los Angeles, CA (US)

(72) Inventor: Gregory J. Papania, Los Angeles, CA (US)

(21) Appl. No.: 16/991,846

(22) Filed: Aug. 12, 2020

Related U.S. Application Data
(60) Provisional application No. 62/885,385, filed on Aug. 12, 2019.

(51) Int. Cl. A61M 21/02; G06F 3/01

(52) U.S. Cl. CPC A61M 21/02; A61M 2021/0027; G06F 3/015

(57) ABSTRACT

The present invention advances the state of mood-inducing music and sound technology by incorporating selected combinations of factors to create recordings, each having specific mood-inducing profiles for a user. Recordings may be stored in novel personal listening devices, such as headsets or earphones, and the selection for play of one recording to induce a desired or appropriate mood may be made by the user using the device itself, an app connected to the device, or may be automatically selected for the user using brain-sensing feedback technology.

### RELATED APPLICATIONS

The present invention claims the benefit of U.S. Provisional Application No. 62/885,385 filed on Aug. 12, 2019.

### TECHNICAL FIELD

The present invention relates generally to the field of acoustical entrainment systems and in particular to systems and methods for providing brain entrainment (“braintrainment”) and mood-inducing sounds and music to such users and other systems.

### BACKGROUND

Systems, devices and methods used to aide in meditation and to bring the brain, body, and psyche into a desired mood state are well known. For example, entrainment, which is synchronization of a human system, such as the brain, to an external perceived rhythm generated by music, dance, beat-inducing instruments, including primitive tools, has been used for millennia by many cultures the world over. Beat induction, in which a regular isochronous pulse is activated while one listens to drums or music, is a common example.

The human brain generates the brainwaves, typically grouped into 5 types, categorized by their frequency ranges. As shown in Table 1, each brainwave type is usually associated with a primary function or brain state:

TABLE 1

| | | |
|---|---|---|
| Frequency | | |
| range | Name | Usually associated with: |

| | | | |
|---|---|---|---|
| >40 | Hz | Gamma waves | Higher mental activity, including |
| | | | perception, problem solving, and |
| | | | consciousness |
| 13-39 | Hz | Beta waves | Active, busy thinking, active |
| | | | processing, active concentration, |
| | | | arousal, and cognition |
| 7-13 | Hz | Alpha waves | Calm relaxed yet alert state |
| 4-7 | Hz | Theta waves | Deep meditation/relaxation, REM |
| | | | sleep |
| <4 | Hz | Delta waves | Deep dreamless sleep, loss of body |
| | | | awareness |

Thus, when isochronic tones are created for brain entrainment, auditory pulsations are generated at a frequency (speed) that matches the brainwave frequency they are connected to. For example, a theta range of 4-7 Hz means the isochronic tone's pulsations would occur in a rhythm that matches the wavelength of frequencies between 4 and 7 Hz, thus creating a “theta isochronic tone.”

Binaural beats, also called binaural tones, is another form of brain entrainment used for mood state therapy. A binaural beat is an auditory illusion perceived when two different pure-tone sine waves, typically both with frequencies lower than 1500 Hz, and with less than a 40 Hz difference between them, are presented to a listener, usually dichotically (one through each ear). For example, if a 530 Hz pure tone is presented to a subject's right ear, while a 520 Hz pure tone is presented to the subject's left ear, the listener will perceive the auditory illusion of a third tone, in addition to the two pure-tones presented to each ear. The third sound is called a “binaural beat”, and in this example would have a perceived pitch correlating to a frequency of 10 Hz, that being the difference between the 530 Hz and 520 Hz pure tones presented to each ear. This would be called an “alpha binaural tone,” 10 Hz being within the 7-13 Hz alpha wave range. The purpose for providing binaural beats having this frequency would be “entrain” those alpha brainwaves of the subject that hover in the 10 Hz range (but perhaps slightly above or below) to precisely 10 Hz. The science of binaural-beat perception is fairly well-understood, but its application for mind and mood therapy, particularly in consumer products, is more recent.

In yet another variation, “bilateral” and “EMDR” music involve methods of panning sounds and tones from side to side—from ear to ear—rhythmically, to induce presence and relaxation due to the brain's reaction to the moving stimuli.

As the speed of modern society increases, the population becomes increasingly distracted, manic, and stressed. Brainwave entrainment is a solution to the detrimental emotional, mental, and physical effects of living in modern society. Accordingly, these techniques have found increasing use and offerings in the growing health and wellness space.

Many studies have shown the effectiveness of binaural and isochronous entrainment on human subjects, and have applied them in many ways, including adjusting frequencies for different effects, and creating tones in various combinations and in conjunction with music. For example, Centerpointe Research Institute, the Monroe Institute and the website http://uazu.net/abagen/ all provide background information on the subject.

Indeed, in study after study, various benefits of brain entrainment have been demonstrated. Following are some statistics with sources identified thereafter. After listening to music designed to induce theta wave production, 78% of subjects were able to enter a meditative state within 5 minutes ⁽¹⁾. After 30 days of listening participants suffering from anxiety were able to cut their symptoms by 50% ⁽²⁾. Subjects who listened to music with delta waves improved their sleep habits by nearly 60% after 30 days ⁽³⁾. Tests conducted with subjects who had headaches showed a 90% reduction in pain within 20 minutes of listening to delta wave music ⁽⁴⁾. Daily usage of beta and gamma wave stimulation showed a 34% reduction in fatigue within 7 weeks ⁽⁵⁾. While listening to music with beta waves subjects showed an 81% improvement in focus and concentration ⁽⁶⁾. Students listening to beta wave music showed grade and learning retention increases 3× greater than average students within 2 weeks' time ⁽⁷⁾. In another study average IQ was shown to increase between 23%-33% and long-term improvements in self-stem, concentration, sleep patterns, irritability, & organization were shown ⁽⁸⁾. Memory improvement increased 15% by subjects listening to alpha wave music while reading, engaging in conversation or seminars, and performing other daily tasks ⁽⁹⁾. Listening to binaural music positively affects the production of hormones directly related to longevity of life, well-being, and stress. Subjects cortisol levels decreased 46%, DHEA increased by 43%, and melatonin production increased by 98%⁽¹⁰⁾. Listening to alpha waves aids in production of serotonin and listening to theta waves helps boost catecholamines, which are vital to memory and learning⁽¹¹⁾. After 13 months, studies inside an AA group who listened to binaural music showed only 20% relapse and depression reduction to 1/3 compared to the average program attendee. ⁽¹²⁾. Brainwave synchronization induced by binaural music—when the left and right brain hemispheres operate in harmony a person thinks more effectively, alertness yet relaxation and peace ensue ⁽¹³⁾.

### SOURCES

(1) Kroger, W. S., & Schneider, S. A. (1969). An electronic aid for hypnotic induction: A pret, immmary report. International Journal of Clinical and Experimental Hypnosis, 7(2), 93-98.
(2) Le Scouamec R P, Poirier R M, Ownes J E, Gauthier J, Taylor A G, Foresman P A. Use of binaural beat tapes for treatment of anxiety: a pilot study of tape preference and outcomes. Altem Ther Health Med. 2001, 7(1):58-63.
(3) Michel Le Van Quyen, et al. “Large-Scale Cortical Dynamics of Sleep Slow Waves.” Plos ONE 7.2 (2012): 1-10; and Slever, D. (2002) “The Rediscovery of Audio-Visual Entrainment Technology.”
(4) Solomon G D. Slow wave photic stimulation in the treatment of headache—a preliminary report. Headache. 1985; 25(8):444-446.
(5) Howard C E, Graham L E, 2nd, Wycoff S J. “A comparison of methods for reducing stress among dental students.” J Dent Educ. 1986;50(9): 542-544.
(6) Patrick G J. “Improved neuronal regulation in ADHD: An application of 15 sessions of photic-driven EEG neurotherapy”. J Neurother. 1996;1(4):27-36.
(7) Joyce M, Siever D. “Audio-Visual Entrainment (AVE) program as a treatment for behavior disorders in a school setting.” J Neurother. 2000;4(2):9-25.
(8) Dr. Siegried Othmer
(9) Williams, J., Ramaswamy, D. and Outhaj, A., 2008.
(10) Dr Vincent Giampapa, MD, Former President of American Board of Anti-Aging Medicine, New England Journal of Medicine.
(11) Dr Margaret Patterson & Dr Ifor Capel, Marie Curie Cancer Foundation Research Department.
(12) Peniston and Kulkosky, 1989.
(13) “On the Frequency Limits of Binaural Beats”, Harvard & Acoustical Society of America.

Conventionally, when used for entrainment or mood effects on humans, binaural and isochronic tones have been composed and recorded on various media and played in the environment of listeners through conventional speakers, such as in a quiet room or used with headsets. Alternatively, numerous binaural beats apps are now available to download from an “app store” onto one's mobile device. Examples include the “Brainwave: 35 Binaural Series” app, the “Binaural (β)” app, and “Binaural Beats,” all available from the Apple's “App Store.” These apps enable a user to generate tones or play pre-recorded tones from their mobile devices and to transmit them to external speakers or to headsets via conventional wired or wireless technology, such as the Bluetooth™ standard.

However, these solutions have several drawbacks. For one, getting binaural and isochronous tones and music into the “hands” of individuals can be challenging. Not everyone is “app” or “tech” savvy or wants to bother with the steps needed to set up these systems for personal use. These people exist in all age groups—however, they mostly exist in the 27 and up groups. Many people simply want to meditate and or change their mindstates and better their lives, but they won't do it if it involves even minimally complicated setups or downloads of what they may term as “strange” music. Second, the conventional solutions of playing these tones in a room, or downloading an app with stored tones and transmitting the tones to a headset via wireless technology are crude and less than ideal. The frequency and tonal qualities of these solutions are often distorted by distance (in the projected sound environment) and imprecise frequency selection in self-controlled systems. Moreover, these solutions are often not ideal for the mass market in that the tones are not professional-tuned to best effectuate the desired outcomes.

Finally, many of these solutions offer pure binaural and isochronic tones and are sometimes perceived as too monotonous and less than pleasant to the listener(s), inappropriate for some environments and deterring some from using these potentially very helpful tones, especially for longer periods of uninterrupted use, such as for hours or overnight.

Accordingly, what is needed are simple, high quality user controllable, dedicated systems and devices that are easy enough for anyone to use while providing high quality, professionally-engineered, binaural and isochronic tones that are pleasant to listen to for short and extended periods of time, resulting in maximum effectiveness for their users.

### SUMMARY

The present invention meets these needs by disclosing systems, methods and products that solve the aforementioned problems and more. The preferred systems and methods involve producing musical tracks layered with high quality, binaural and isochronous tones, storing them electronically on dedicated personal listening devices, such as over-the-ear and in-ear headphones. The resident music tracks are preferably custom-tailored to each desired mood state, with the tones created preferably in the 432 Hz and 444 Hz tunings. Having these high-quality tones overlaid-with-music tracks played near or in the subject ear canals effectively eliminates distortion and other quality problems. Not having to rely on an external source for this audio content simplifies operation and use and eliminates the need to carry around a mobile device to pair with earphones or headphones. With the present invention, one simply places the inventive, dedicated, mood-inducing, or mindset, headphone or earphone set on or in his/her ears, powers up, and selects and plays a selected pre-recorded track that matches and induces the user's desired balanced state.

The present invention operates with either headphones that include the tones and music stored in dedicated memory, or can be used with in-the-ear earphones that likewise can store the recordings or receive them from an external source.

In one embodiment, a system for enhanced brain entrainment includes a headset or earphones (the device) in conjunction with a companion app on a mobile device (the app) that pairs with the device using any known connection means, including standard Bluetooth technology.

What is also needed is an improvement in headset entrainment technology that enables even better frequency matching of a user's brainwave patterns to a desired state. Accordingly, the present invention also discloses the alternative embodiment of a “brain sensing headphone feedback system” having an over-the-head headphone as discussed above (any embodiment) but modified to include strategically-placed brain sensors incorporated therein and feedback processing, When the headphone is placed on the head of a user and turned on, the sensors are strategically positioned at various spots on the head in order to sense the user's brainwaves—brain frequency activity—, either while playing tones and/or music or in a quiet state. The sensors send these brainwave signals to the processor for feedback processing. The processor can be programmed to process these signals in a number of useful ways. The feedback signals may be stored locally (on the headset) or sent to remote device, such a mobile device connected to the headset via an app for storage and further processing, and potentially for later analysis. In another embodiment, the brainwave feedback signals are processed in real time in order to detect brain frequency activity that is inconsistent with the desired result. In such case, the system may send real-time feedback to the user. In another embodiment, the sensing processor can be programmed to provide a signal back to the system itself indicating that the track the tones and/or music—being played for the user is not effectively entraining the user. In such case the processor will automatically adjust the tones/music to a different frequency or tonal quality that better suites the user desiring entrainment for a particular mood or mind set.

It is to be understood that the invention is not limited in its application to the details of construction and the arrangement of components described hereinafter and illustrated in the drawings and photographs. Those skilled in the art will recognize that various modifications can be made without departing from the scope of the invention.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a perspective view of an over-the-ear headphone system showing many components and features in accordance with one non-limiting embodiment of the present invention; and

FIG. 2 is a perspective view of an in-the-ear earphone system showing internal components in accordance with one non-limiting embodiment of the present invention; and

FIG. 3 is a flow diagram of an exemplary process according to the present invention for creating, recording and storing mood-inducing recordings on a user device, along with options presenting selected recordings to the device.

### DETAILED DESCRIPTION OF THE PREFERRED EMBODIMENTS

Referring now to the drawings, like reference numerals designate identical or corresponding features throughout the several views.

The present invention discloses a method for providing brainwave entrainment that preferably comprises the steps of electronically recording 102 one or more sound recordings comprising at least one entrainment characteristic selected to induce a specific mood; electronically storing 104 the recording on a listening device; and making 106 the stored recording playable by a user of the device.

The recorded entrainment characteristic is preferably selected from any one or more of a selected grounding tone on the “Solfeggio scale”, a musical tuning frequency detuned from the standard A-scale 440 Hz tuning frequency, namely 432 Hz and 444 Hz, and an entrainment technique. The entrainment technique may be either a binaural technique or isochronic technique.

The present invention also discloses a brain entrainment system comprising a personal listening device; and a brain entrainment recording electronically stored in and selectably played by the device. The recordings preferably incorporate at least one entrainment characteristic selected to induce a specific mood. The personal listening device may be an over-the-ears headset or in an in-the-ear earphone. The system may further include an app in communication with the personal listening device to control the play of the recordings in the device.

The present invention also discloses a brain entrainment, mood-inducing headset system, that includes a headset for a user, brain entrainment recordings electronically stored in the headset, each recording tuned to induce a different mood state; an an electronic brainwave feedback subsystem comprising one or more brain sensors for placement on the head of the user and adapted to sense electrical brain activity of the user; and a feedback circuit in the headset in electrical communication with the one or more sensors adapted to read the sensed electrical brain activity, wherein the headset is programmed to automatically select and play a recording based on the sensed brain activity.

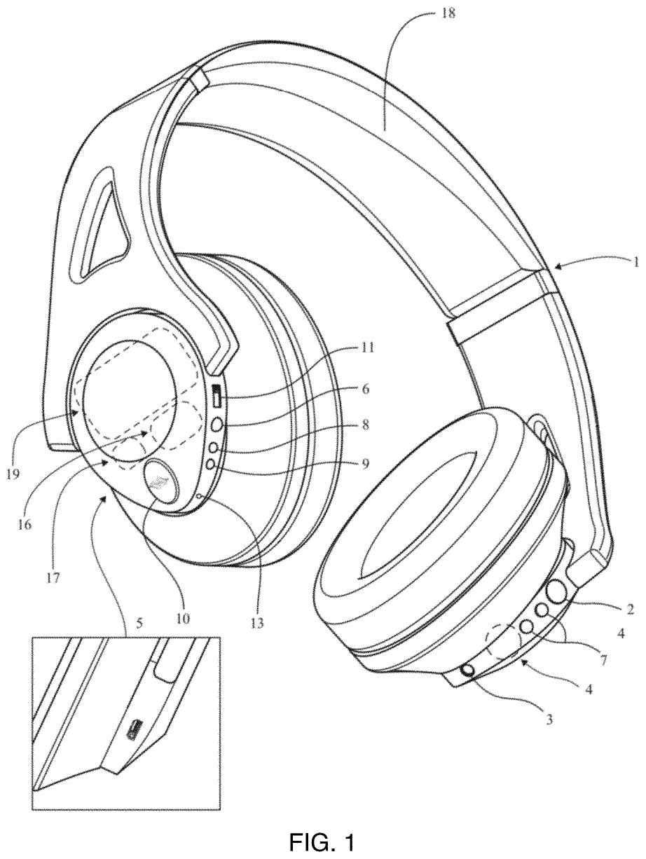

In one preferred embodiment, a device in accordance with the present invention is shown in FIG. 1. The device 1 is a computer-controlled, battery-powered, programmable over-the-ear headset. In particular, the device includes noise-canceling (ANC) activated by button 2, a 3.5 mm audio jack 3, Bluetooth connectivity for wireless use and micro USB connectivity 5. The headset 1 includes many conventional features, such as a play/pause button 6, volume control buttons 7, forward and reverse buttons 8, 9 for moving through music tracks. The inventive system also includes a built-in mood-selection button 10 that by repeatedly pressing moves through different mood inducing tracks as explained further below. The device preferable also includes a sliding power button/Bluetooth pairing (when held down) 11, a built-in microphone 12 for communications, an on-board sound processor and MP3 player 13, and on-board storage for mood music (not shown), The headset also includes memory foam head padding 15 for extended periods of use.

In this embodiment, the present invention versatilely functions as a stand-alone headset capable of playing brain tones or serving as a noise-cancellation headset, or connecting to a phone and play music from other apps. It also has the ability to turn off the incoming phone call feature 17 (which can be done through a companion app). It should also be understood that the professionally procured music can be updated via an app via Bluetooth. Preferably, each music piece (“track” will play for approximately 20 mins and then fade into another track in the same mood category, for a total of 3 to 7 hours-worth of pre-loaded music per mood.

An important component of the present invention and of device 1 is the mood music provided. The inventor has discovered that certain combinations of (a) specific grounding tones on the “Solfeggio scale”, (b) one or both of two musical tuning frequencies detuned from the standard A-scale 440 Hz tuning frequency, namely 432 Hz and 444 Hz, and (c) entrainment techniques (binaural or isochronic, or both) provide enhanced and even optimum mood creating or mood-altering experiences. Table 2 provides one set of preferred frequency ranges identified by the inventor for achieving various moods.

TABLE 2

| Desired Mood/ | Brainwave | Solfeggio | Brainwave | Tuning | |
|---|---|---|---|---|---|
| Mindset | Range | Tone | States | Frequency | |
| Sleep | .2 hz to 8 hz | 174 Hz | Delta, theta; | 432 | Hz scale |
| | | | binaural | | |
| Relax/Meditate | 4 hz to 13 hz | 417 Hz | Theta, alpha; | 432 | Hz scale |
| | | | binaural | | |
| Focus | 10 hz to 18 hz | 852 Hz | Alpha, beta; | 444 | Hz scale |
| | | | binaural, | | |
| | | | isochronic | | |
| Calm/Balance/ | 8.5 hz to 14 hz | 639 Hz | Alpha; binaural, | 432 | Hz scale |
| De-Stress | | | isochronic | | |
| Create | 4 hz to 13 hz | 528 Hz | Theta, alpha; | 444/432 | Hz scales |
| | | | binaural | | |
| Energy | 15 hz to 50 hz+ | 963 Hz | Gamma, high- | 444 | Hz scale |
| | | | beta; binaural | | |
| Joy | 8 hz to 13 hz | 396 Hz | Alpha; binaural | 444 | Hz scale |

Referring to the first row of Table 2—Sleep—According to scientific research and sleep studies, human brains fluctuate between delta and theta states during sleep. Thus, a human using reproductions of brainwave frequencies via audio would listen to frequencies in such frequency ranges to induce the desired effect. 174 Hz is a tone on the solfeggio scale which the inventor has found to be particularly effective for grounding as it is a low pitch with long wavelengths. Moreover, 432 Hz was selected as a musical tuning frequency for the musical composition that intertwines with the tones. Detuned from the standard A-440 Hz for music (e.g., guitar) to 432 Hz tuning is believed to induce relaxation due to the longer wavelengths produced by instruments that are playing in a more relaxed state. For example, when you detune a string instrument the strings are more relaxed. In contrast, 444 Hz is a musical tuning that is sharp and brighter than A-440. When listening to music in 444 Hz, the feeling is one of joy and engagement given that the instruments sound brighter and the wavelengths are shorter than standard A-440 tuning.

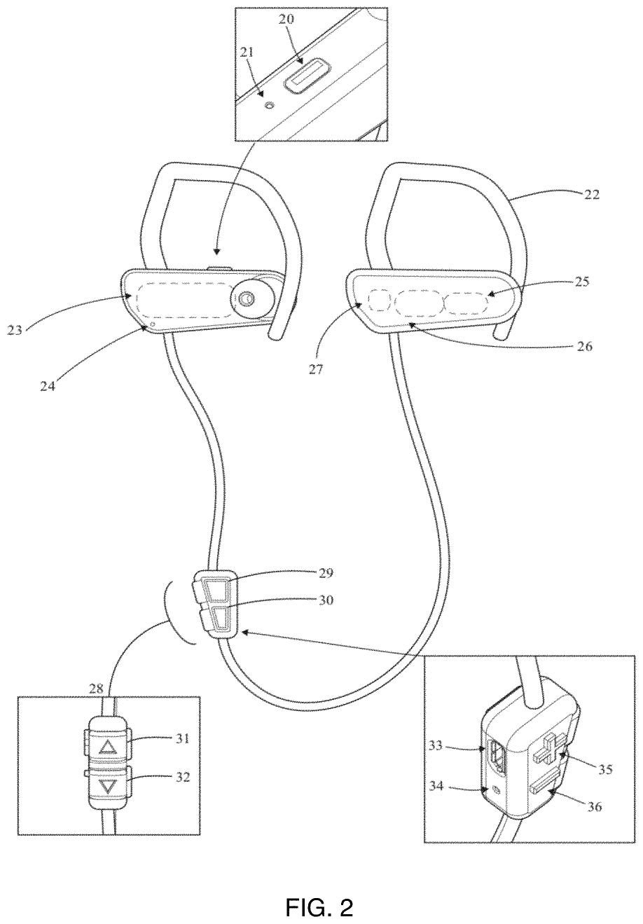

Turning back to the devices and systems of the present invention, in another embodiment, the mood music is transmitted to the user via in-the-ear earphones. An example of such a device is shown in FIG. 2. Similar to the over-the-ear embodiment, this embodiment may include noise canceling, Bluetooth enabled (BTLE), wire enabled, onboard MP3 player with pre-loaded brain entrainment/mood music layered with binaural, isochronic, and solfeggio tones separated by “moods” (Sleep, Relax/Meditate, Focus, Balance/De-stress, Create, Joy, Energy) available to the user via selection buttons. Turning to FIG. 2., the earbuds in the preferred embodiment of the present invention connect to an app via BTLE that can track meditation progress and be used to update music on headphones. Additionally, the in-ear earphones or earbud include (20) Power button/Bluetooth pairing; 21. Power/Bluetooth indicator LED; 22. Bendable over-ear wire; 23. Internal rechargeable Lipo battery; 24. Microphone; 25. On-board storage for mood music; 26. On-board sound processor/MP3 player chip; 27. Bluetooth Low Energy antenna (BTLE); 28. Tethered control remote; 29. Play/pause button; 30. Mood selection button; 31. Forward button for music tracks; 32. Back button for music track; 33. Micro USB charging port; 34. Charging indicator LED; 35. Raise volume button; 36. Lower volume button; 37. Noise cancelling (ANC); 38. Functions as stand-alone headphones to play brain tones or noise-cancellation, or connect to phone and play music from other apps; 39. Ability to turn off the incoming phone call feature (can be done through app); and 40. Music can be updated via the app via Bluetooth—each music piece will play for approximately 20 minutes and then fade into another in the same category for 3 to 7 hours-worth of pre-loaded music per mood.

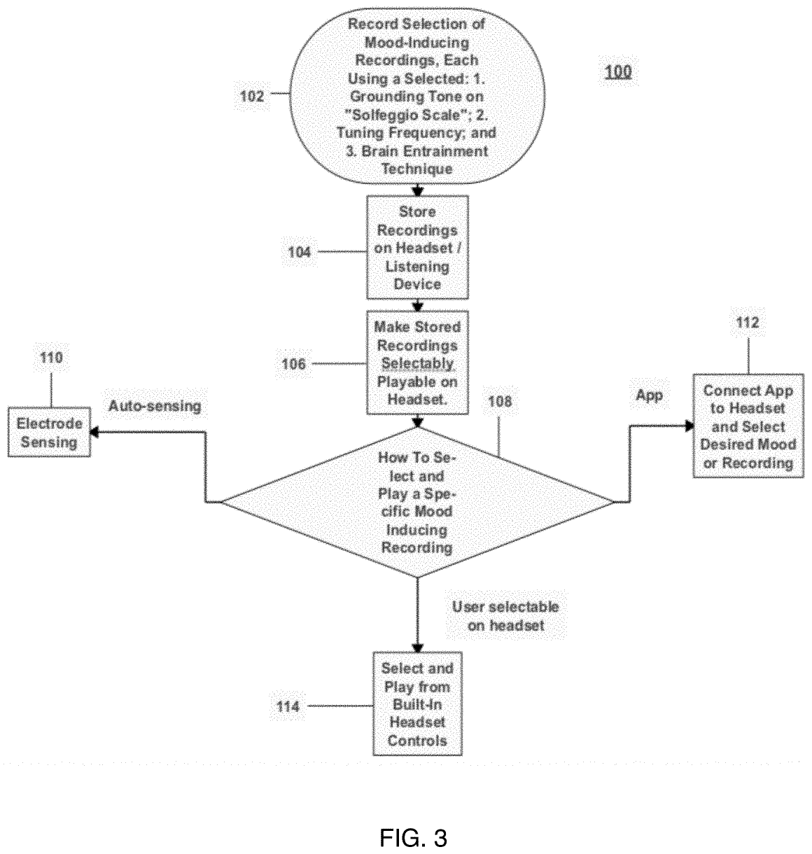

Methods of the present invention may be implemented as shown in FIG. 3. As seen, in step 102, mood inducing recordings are recorded and selected using any one or more of the criteria described above. In step 104, the recordings are store on a listening device, such as the headset discussed above. In step 106, these recordings can be played on the headset by selecting, or having selected for a user, a desired or appropriate recording. This can be achieved in any number of ways. As seen in decision tree 108, any one or more of three selection options may be presented to a user. At step 114, the user may select a recording of interest from a library of recordings stored on the device by manually pressing controls on the listening device on the head. Alternatively, at step 112 the user may select either a desired mood recording or select a specific recording on an app on user's remote device, such as a mobile device, that is wirelessly connected to the listening device (more on this below). Or, in step 110, a mood-inducing recording may be automatically selected for the user using automatic mood-sensing technology. This technology comprises mood-sending electrodes that are place on the head the user, and a specially programmed processor/controller that (a) interprets the user's electrical “mood state” brain activity from the received electrode signals and (b) automatically selects an appropriate recording relative to the sensed state. These components may be integral with the headset or may be an add-on to the headset or earphones.

As noted above. the present invention can also be implemented with the aid of a mobile app, as seen at step 112 of FIG. 3. In such an embodiment, headset or headphone device connects to a user's mobile phone via Bluetooth, or other known means and sends the music (or beats) to the device. At this time, the device may also receive settings changes from the app. Additional features of the app may include: the ability to update mood music to headsets; processing brain activity monitoring when paired with the brain sensing headphones, a “do-not-disturb” feature to stop phone calls and other notifications from entering the headphones; the ability to track meditation and other mood progress, including how often and how long; a “Project tracker” that tracks and displays how long a user has been working on a project; ability to set meditation, focus, or productivity goals; reminder functionality, to remind users to take needed breaks or stay motivated to keep working to reach set goals; a time limiter to set specific amounts of time to meditate, relax, or focus on another task or mood and to alert the user when the goal has been reached; an export function so that work time can be exported from the app and used by trainers to track project timing for their own clients or for oneself. Further, in over-the-ear headphones with brain sensing technology incorporated therein, app functionality may expand to track and show brainwave statistics and to give users listening suggestions based on their recorded brainwaves.

In alternative embodiments, the mood music of the present invention may be stored on miniaturized mp3 type players that are pendant-sized, small enough for example to fit comfortably on a neck strap or in a pocket. The mood music may also be stored on a watch such as an iWatch and the like, or device such as a Fitbit and the like.

While embodiments of the invention have been illustrated and described, it is not intended that these embodiments illustrate and describe all possible forms of the invention. Various changes, modifications, and alterations in the teachings of the present invention may be contemplated by those skilled in the art without departing from the intended spirit and scope thereof. For example, the disclosures herein, including but not limited to the novel music-overlaid, entrainment beats, may be used in many environments and industries such in as the healthcare and therapy (physical and mental), entertainment, education, travel, fitness, office, agriculture and retail industries. The solution disclosed herein also may be incorporated into many products and services, including but not limited to “smart speakers”, children and baby sound products, live show installations, spas, agricultural/growing products and many other products and services. It is intended that the present invention encompass such changes and modifications.

What is claimed is:

1. A method for providing brainwave entrainment, comprising:
a. electronically recording a sound recording comprising at least one entrainment characteristic selected to induce a specific mood;
b. electronically storing the recording on a listening device; and
c. making the stored recording playable by a user of the device.

2. The method of claim 1, wherein the recorded entrainment characteristic is selected from any one or more a selected grounding tone on the “Solfeggio scale”, a musical tuning frequency detuned from the standard A-scale 440 Hz tuning frequency, namely 432 Hz and 444 Hz, and an entrainment technique.

3. The method of claim 1, wherein the entrainment technique is one of a binaural technique or isochronic technique.

4. The method of claim 1, wherein the recording is recorded using all of a selected grounding tone on the Solfeggio scale, a selected musical tuning frequency detuned from the A-scale 440 Hz tuning frequency and a selected entrainment technique.

5. A method for providing brainwave entrainment, comprising:
a. electronically recording two or more sound recordings, each recorded with a selected grounding tone on the Solfeggio scale, and a selected musical tuning frequency detuned from the A-scale 440 Hz tuning frequency, all selected to induce a specific mood;
b. electronically storing the recording on a listening device; and
c. making the stored recording selectably playable by a user of the device.

6. The method of claim 6, wherein at least one of the recordings is further recorded using a selected entrainment technique.

7. A brain entrainment system comprising:
a. a personal listening device; and
b. a brain entrainment recording electronically stored in and selectably played by the device.

8. The brain entrainment system of claim 8, wherein the recording incorporates at least one entrainment characteristic selected to induce a specific mood.

9. The brain entrainment system of claim 8, wherein the personal listening device is an over-the-ears headset.

10. The brain entrainment system of claim 8, wherein the personal listening device is an in-the-ear earphone.

11. The brain entrainment system of claim 8, further including an app in communication with the personal listening device to control the play of the recordings in the device.

12. A brain entrainment, mood-inducing headset system, comprising:
a. a headset for a user;
b. brain entrainment recordings electronically stored in the headset, each recording tuned to induce a different mood state; and
c. an electronic brainwave feedback subsystem comprising
i. one or more brain sensors for placement on the head of the user and adapted to sense electrical brain activity of the user; and
ii. a feedback circuit in the headset in electrical communication with the one or more sensors adapted to read the sensed electrical brain activity;
wherein the headset is programmed to automatically select and play a recording based on the sensed brain activity.
